(19) United States
(12) Patent Application Publication
(10) Pub. No.: US 2022/0015951 A1
(43) Pub. Date: Jan. 20, 2022

(54) BANDAGE WITH REPLACEABLE TREATMENT MEMBER

(71) Applicant: BandGrip, Inc., Chicago, IL (US)

(72) Inventor: Fred Smith, Houston, TX (US)

(21) Appl. No.: 17/379,947

(22) Filed: Jul. 19, 2021

Related U.S. Application Data
(60) Provisional application No. 63/053,653, filed on Jul. 19, 2020.

(51) Int. Cl. A61F 13/02

(52) U.S. Cl. CPC A61F 13/0236; A61F 13/0266

(57) ABSTRACT

A bandage and method of coupling the bandage to a skin of a patient. The method comprises placing an opening of a bandage over a wound. The bandage comprises a base substrate, a mechanical coupling system, and the opening. The base substrate includes an inner surface, an outer surface, a first side edge, and a second side edge. The mechanical coupling system includes a first mechanical coupling member disposed proximate to the first side edge of the base substrate and a second mechanical coupling member disposed proximate to the second side edge of the base substrate. The first and second mechanical coupling members each includes a plurality of gripping structures to hold a wound together. The opening is disposed through the base substrate, the opening being also disposed between the first and second mechanical coupling members.

### CROSS-REFERENCE TO RELATED APPLICATION

This application claims priority from U.S. Provisional Pat. App. No. 63/053,653, entitled “BANDAGE WITH REPLACEABLE TREATMENT MEMBER”, filed Jul. 19, 2020, the entire specification of which is hereby incorporated by reference in its entirety.

This application is related to U.S. patent application Ser. No. 15/349,484, entitled “Bandage”, filed Nov. 11, 2016, and U.S. patent application Ser. No. 15/801,529, entitled “Bandage And Anchor for Bandages”, filed Nov. 2, 2017, while not claiming priority from either, and, the entire disclosures of each of which are hereby incorporated by reference in their entireties.

### BACKGROUND OF THE DISCLOSURE

### 1. Field of the Disclosure

The disclosure relates in general to the treatment of wounds, and more particularly, to a bandage that can be placed over wounds that also aids in the closure of a wound, as well as an anchor for bandages that can aid in the maintenance of a bandage or closure in the proper orientation.

### 2. Background Art

The use of bandages is well known in the art. Generally, bandages include a base substrate, the base substrate being adhered to the skin of a user. Often, a wound that is deep may require stitches or the like to provide the necessary closure. Problematically, a typical bandage while helpful, often does not provide any, or sufficient force on a wound to direct the opposing sides of the wound to meet or to otherwise foster closure. Some bandages have been developed that include structures that engage the epidermis and the dermis so as to anchor portions of the bandage thereto. While such bandages have been developed, many of such bandages lack sufficient retaining strength to be anchors, or, are excessively intrusive and can, themselves lead to injury and infection.

### SUMMARY OF THE DISCLOSURE

The disclosure is directed to a bandage comprising a base substrate, a mechanical coupling system, and an opening. The base substrate includes an inner surface, an outer surface, a first side edge, and a second side edge. The mechanical coupling system includes a first mechanical coupling member disposed proximate to the first side edge of the base substrate and a second mechanical coupling member disposed proximate to the second side edge of the base substrate. The first and second mechanical coupling members each includes a plurality of gripping structures to hold a wound together. The opening is disposed through the base substrate, the opening being also disposed between the first and second mechanical coupling members.

In at least one configuration, the bandage further comprises a third mechanical coupling member disposed proximate to the first side edge of the base substrate and a fourth mechanical coupling member disposed proximate to the second side edge of the base substrate.

In at least one configuration, the opening through the base substrate is comprised of a mesh strip.

In at least one configuration, the mesh strip is pre-impregnated with a skin adhesive, the bandage further comprising a back side liner disposed on the mesh strip to prevent the pre-impregnated skin adhesive from being activated.

In at least one configuration, the opening through the base substrate includes a first column of a plurality of openings and a second column of the plurality of openings.

In at least one configuration, the first column of the plurality of openings is staggered with respect to the second column of the plurality of openings.

In at least one configuration, the opening through the base substrate includes a third column of the plurality of openings.

In at least one configuration, the opening is rectangular in shape.

In at least one configuration, the opening is circular in shape.

In at least one configuration, the bandage further comprises a replaceable treatment member coupled to the base substrate, the replaceable treatment member including a wound covering that corresponds to the opening, the wound covering to cover the wound.

In at least one configuration, the bandage further comprises a skin adhesive disposed on an outer surface of the base substrate.

In at least one configuration, the bandage further comprises an adhesive covering liner disposed on the base substrate.

In at least one configuration, the first and second mechanical coupling members each includes rows of the plurality of gripping structures.

In at least one configuration, the base substrate is approximately 2 inches in width and 3 inches in height.

In at least one configuration, the opening includes a plurality of columns of openings, and the bandage further comprises a mesh strip disposed on the plurality of openings and on a same side of the base substrate as the mechanical coupling system.

In at least one configuration, the bandage further comprises an adhesive delivery system to store a skin adhesive and deliver the skin adhesive to the openings through the base substrate.

The disclosure is also directed to method of coupling a bandage to a skin of a patient. The method comprises placing an opening of a bandage over a wound. The bandage comprises a base substrate, a mechanical coupling system, and the opening. The base substrate includes an inner surface, an outer surface, a first side edge, and a second side edge. The mechanical coupling system includes a first mechanical coupling member disposed proximate to the first side edge of the base substrate and a second mechanical coupling member disposed proximate to the second side edge of the base substrate. The first and second mechanical coupling members each includes a plurality of gripping structures to hold a wound together. The opening is disposed through the base substrate, the opening being also disposed between the first and second mechanical coupling members.

In at least one configuration of the method, the bandage further comprises a third mechanical coupling member disposed proximate to the first side edge of the base substrate and a fourth mechanical coupling member disposed proximate to the second side edge of the base substrate.

In at least one configuration, the opening through the base substrate is comprised of a mesh strip.

In at least one configuration, the mesh strip is pre-impregnated with a skin adhesive, the bandage further comprising a back side liner disposed on the mesh strip to prevent the pre-impregnated skin adhesive from being activated, the method further comprising pulling back the back side liner to activate the pre-impregnated skin adhesive.

### BRIEF DESCRIPTION OF THE DRAWINGS

The disclosure will now be described with reference to the drawings wherein:

FIG. 1 illustrates a first side view of an example bandage, in accordance with at least one configuration disclosed herein;

FIG. 2 illustrates a second side view of the bandage shown in FIG. 1, in accordance with at least one configuration disclosed herein;

FIG. 3 illustrates a bottom edge view of the bandage shown in FIG. 1, in accordance with at least one configuration disclosed herein;

FIG. 4 illustrates a second side edge view of the bandage shown in FIG. 1, in accordance with at least one configuration disclosed herein;

FIG. 5 illustrates an isometric view of example peelable bandage coverings of the bandage shown in FIG. 1, in accordance with at least one configuration disclosed herein;

FIG. 6 illustrates an isometric view of the bandage shown in FIG. 1, in accordance with at least one configuration disclosed herein;

FIG. 7 illustrates a top plane view of an example mechanical coupling system showing, in particular, an upstanding structure, in accordance with at least one configuration disclosed herein;

FIG. 8 illustrates a cross sectional view of an example gripping structure, in particular, the base configuration thereof, in accordance with at least one configuration disclosed herein;

FIG. 9 illustrates a partial perspective view of the gripping structure showing, in particular, the upstanding structure, in accordance with at least one configuration disclosed herein;

FIG. 10 illustrates a perspective view of the gripping structure showing, in particular, an upstanding structure, in accordance with at least one configuration disclosed herein;

FIG. 11 illustrates a perspective view of the gripping structure showing, in particular, an upstanding structure, in accordance with at least one configuration disclosed herein;

FIG. 12 illustrates a cross sectional view of the gripping structure taken generally about lines 12-12 of FIG. 7, showing, in particular, an upstanding structure and the tip, in accordance with at least one configuration disclosed herein;

FIG. 13 illustrates a perspective cross sectional view of the gripping structure taken generally about lines 13-13 of FIG. 7, showing, in particular, the overhanging portion, in accordance with at least one configuration disclosed herein;

FIG. 14 illustrates a perspective cross sectional view of the gripping structure taken generally about lines 14-14 of FIG. 7, showing, in particular, the overhanging portion, in accordance with at least one configuration disclosed herein;

FIG. 15 illustrates a partial side elevational view of another mechanical coupling member, showing, in particular, the configuration of the gripping structure, in accordance with at least one configuration disclosed herein;

FIG. 16 illustrates a partial back elevational view of the mechanical coupling member shown in FIG. 15, showing, in particular, the configuration of the gripping structure, in accordance with at least one configuration disclosed herein;

FIG. 17 illustrates a photograph showing a perspective view of a gripping structure shown in FIGS. 15 and 16, in accordance with at least one configuration disclosed herein;

FIG. 18 illustrates a first side view of another example bandage, in accordance with at least one configuration disclosed herein;

FIG. 19 illustrates the bandage shown in FIG. 18 coupled to the replaceable treatment member shown in FIG. 2, in accordance with at least one configuration disclosed herein;

FIG. 20 illustrates a perspective view of the bandage shown in FIG. 18 with a liner coupled thereto, in accordance with at least one configuration disclosed herein;

FIG. 21 illustrates another perspective view of the bandage shown in FIG. 18 with the liner coupled thereto, in accordance with at least one configuration disclosed herein;

FIG. 22 illustrates a perspective view of yet another example bandage, in accordance with at least one configuration disclosed herein;

FIG. 23 illustrates the bandage shown in FIG. 22 with a back side liner disposed over a mesh strip of the bandage, in accordance with at least one configuration disclosed herein;

FIG. 24 illustrates the bandage shown in FIG. 23 with the back side liner pulled back to expose the mesh strip, in accordance with at least one configuration disclosed herein;

FIG. 25 illustrates a perspective view of even yet another example bandage, in accordance with at least one configuration disclosed herein;

FIG. 26 illustrates a perspective outer surface view of the bandage shown in FIG. 25, in accordance with at least one configuration disclosed herein;

FIG. 27 illustrates an example adhesive delivery system, in accordance with at least one configuration disclosed herein;

FIG. 28 illustrates the adhesive delivery system, shown in FIG. 27, that is couplable to a bandage, in accordance with at least one configuration disclosed herein;

FIG. 29 illustrates a detailed view of the adhesive delivery system shown in FIG. 27, in accordance with at least one configuration disclosed herein; and

FIG. 30 illustrates a mesh strip, shown in FIG. 25, used with the bandage shown in FIG. 29, in accordance with at least one configuration disclosed herein.

### DETAILED DESCRIPTION OF THE DISCLOSURE

While this disclosure is susceptible of configuration in many different forms, there is shown in the drawings and described herein in detail a specific configuration(s) with the understanding that the present disclosure is to be considered as an exemplification and is not intended to be limited to the configuration(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

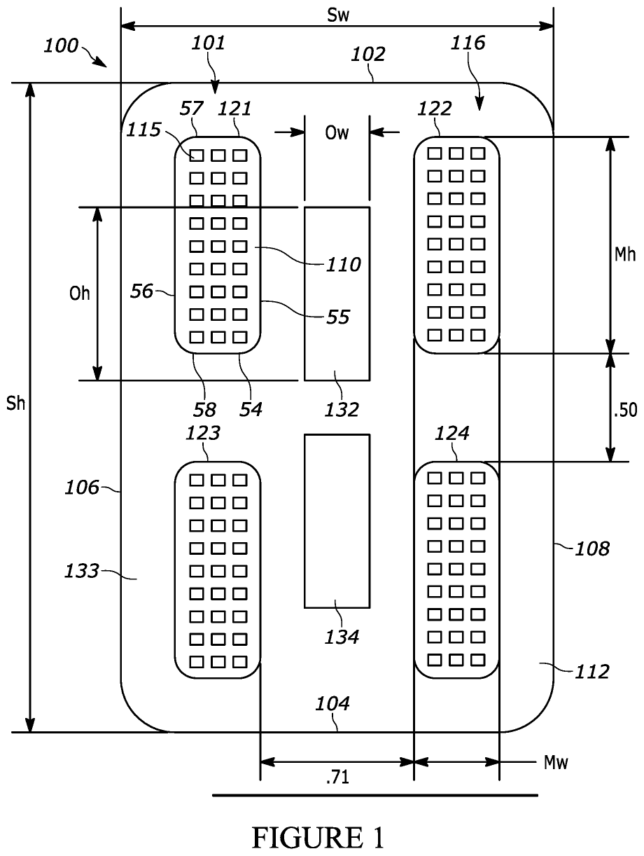

Referring now to the drawings and in particular to FIG. 1, a bandage is disclosed, such as bandage 100. The bandage 100 comprises a base substrate 101, a mechanical coupling system 116, and at least one hole or opening through the base substrate 101, such as holes or openings 132 and 134, respectively. In at least one configuration, the bandage 100 is approximately (+−5%) 3.0 in height Sh and approximately (+−5%) 2.0 inches in width Sw, but one skilled in the art would understand that the bandage 100 can be any size, that is sized to correspond to a wound (not shown) within a skin of a patient (not shown) being treated.

The base substrate 101 is sized in such a way that allows the mechanical coupling system 116 to be relatively positioned and maintained. The base substrate 101 comprises a first end 102, a second end 104, a first side edge 106, a second side edge 108, an inner surface 112, that is a surface that comes into contact with the skin of the patient during use of the bandage 100, and an outer surface 114, that is a surface opposite the inner surface 112 that does not come into contact with the skin of the patient during use of the bandage 100. The first end 102 and the second end 104 are generally opposite and parallel with reference to one another. The first side edge 106, the second side edge 108, the inner surface 112, and the outer surface 114 extend from the first end 102 to the second end 104.

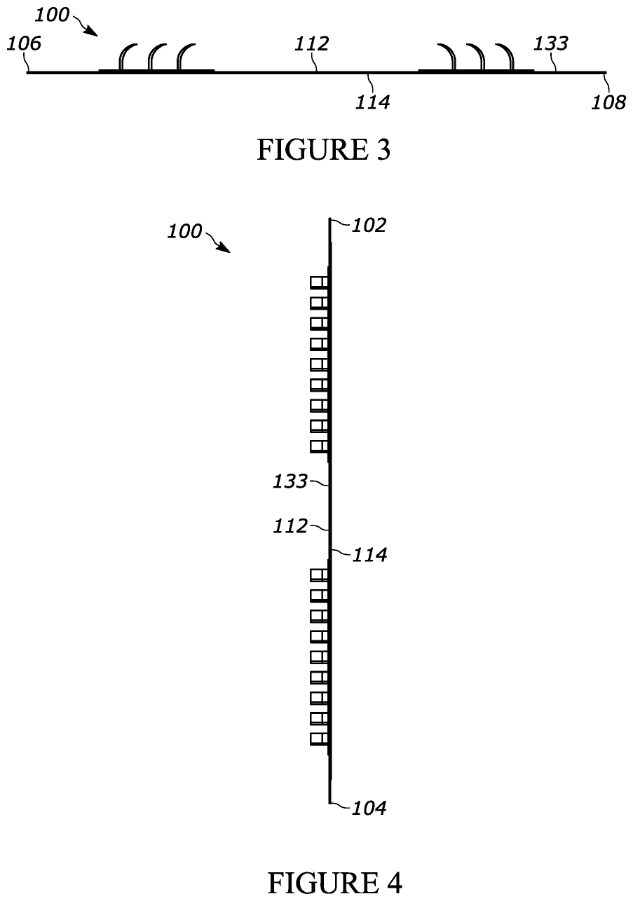

As shown, the angle between first side edge 106 and the second end 104 is approximately (+−10%) 90 degrees, and the angle between the first side edge 106 and the first end 102 is approximately (+−10%) 90 degrees, although other angles are possible. In at least one configuration, the first and second ends 102/104 are approximately (+−5%) 2 inches in width Sw, and the first and second side edges 106/108 are approximately (+−5%) 3 inches in height Sh, although other dimensions are possible for treatment of different side wounds. In at least one configuration, the base substrate 101 includes rounded corners where the first side edge 106 meets the first end 102, where the first end 102 meets the second side edge 108, where the second side edge 108 meets the second end 104, and where the second end 104 meets the first side edge 106, as shown. It is understood by one skilled in the art that the configuration of the base substrate 101 can comprise additional different configurations, including, but not limited to a square, a triangular, circular, and any other shape that contours to various parts of a body of a patient. Some exemplary configurations of the base substrate 101 (with a suitably oriented mechanical coupling system) are shown in FIGS. 1-4 and 6. These are intended solely to be exemplary, and the disclosure is not limited to the configurations shown.

The base substrate 101 can be made of any suitably elastic material such that the bandage 100, such that elastic deformation is allowable to an extent that allows substantial alteration of the initial dimensions of the base substrate 101. Elastic qualities in materials are known to be materials that can deform a given dimension, such as the length, but still possessing the ability to return to its initial dimension without harm to the structure of the material. In this case, an elastic material for base substrate 101 is offering allowance for the dimensions of the base substrate 101, including but not limited to the length and width, to be altered and leaving room for the bandage 100 to attempt to return to its original dimensions. Such an elastic material can further provide an inward biasing force once installed on a patient so as to further aid in the closure of the wound (with the mechanical coupling, at least in part, resisting the biasing force). Materials contemplated include polymer films, natural or synthetic loose fibers that extend between the mechanical coupling members, as well as woven fabric materials, among others. The disclosure is not limited to any particular type of material from which the base substrate 101 can be formed. In some configurations, the base substrate 101 can have limited elasticity in one or more directions.

The mechanical coupling system 116 includes a plurality of mechanical coupling members 121/122/123/124. Although four (4) mechanical coupling members 121/122/123/124 are shown, one skill in the art would understand that the bandage 100 can include more or less mechanical coupling members, dependent upon a size of an area of the skin being treated by the bandage 100. The bandage 100 includes the mechanical coupling members 121/123 disposed proximate to the first side edge 106 and spaced approximately (+−5%) 0.5 inches from each other, and includes the mechanical coupling members 122/124 disposed proximate to the second side edge 108 and spaced approximately (+−5%) 0.5 inches apart from each other. In at least one configuration, the mechanical coupling members 121/122/123/124 are approximately (+−5%) 0.395-0.4 inches in width Mw and are approximately (+−5%) 1.0 inches in height Mh. In at least one configuration, the mechanical coupling members 121/123 are approximately (+−5%) 0.71 inches from the mechanical coupling members 122/124, respectively.

Due to the elasticity of the base substrate 101 and to the elastic stretching of the base substrate 101 over the wound prior to the attachment of the mechanical coupling members 121/123 (or the mechanical coupling members 122/124 depending upon which side of the bandage 101 is first attached to the skin) to the skin, when coupled the base substrate 101 will exert a biasing force to return to its original configuration. As the base substrate 101 attempts to return to its original unstretched configuration, gripping structures 115 resist such movement with a skin adhesive both precluding such movement and also precluding the retraction of the gripping structures 115 from the skin. As such, the gripping structures 115 that extend into the skin on either side of the wound force the skin toward the wound so that the biasing force exerted by the base substrate 101 forces the wound closed, and a general pinching of the skin. Such an operation fosters wound closure, blood clotting, and minimization of scarring. Further, the same is achieved without increasing pain or without causing further damage to the skin around the wound.

Each of the mechanical coupling members 121/122/123/124 includes a base member 110 that is fixed (e.g., glued) to the base substrate 101. The base member 110 is shown as being rectangular with rounded corners, but one skilled in the art would understand that the base member 110 can be other shapes, such as triangular, square, circular, or any other shape. Affixed to the base member 110 are the plurality of gripping structures 115, discussed in more detail below. In at least one configuration, the base member 110 and the gripping structures 115 are integrally formed, such as via injection molding of a polymer (e.g., Lexan®). In at least one configuration, each of the mechanical coupling members 121/122/123/124 include twenty-seven (27) gripping structures 115, three rows of nine (9) gripping structures 115. In at least one other configuration, depending upon the amount of gripping needed for a particular wound, the bandage 100 can include more or less gripping structures 115 than that shown and/or can include more or less rows of gripping structures 115 than that shown.

Disposed approximately (+−5%) centrally between the mechanical coupling members 121 and 122, and centrally between mechanical coupling members 123 and 124 is at least one opening through the substrate 101, such as openings 132 and 134 that are placed over a wound of a patient during use of the bandage 100. In at least one configuration, the openings 132 and 134 are disposed approximately (+−5%) near a middle of the bandage 100, such as approximately (+−5%) 1.5 inches from the first end 102 or the second end 104. Depending upon a size of the bandage 100, the bandage 100 can include more or less openings than those shown, as would be understood by one skilled in the art. In at least one configuration, the openings 132/134 are rectangular in shape with their long axis running along a height of the bandage 100, as shown, although other shapes are possible such as circular, square, oval, triangular, or any other shape. In at least one configuration, the openings 132 and 134 are approximately (+−5%) 0.80 in height Oh and 0.30 in width Ow, although other shapes are possible such as circular, square, triangular, or any other shape. Depending upon a size of the bandage 100, use of a plurality of openings, such as openings 132/134, assists the bandage 100 in maintaining its shape when being stretched.

In at least one configuration, the bandage 100 further includes a skin adhesive 133, such as cyanoacrylates skin adhesive, on the inner surface 112 of the base substrate 101 to allow the bandage 100 to adhere to the skin of the patient. Prior to the bandage 100 being placed on the skin of the patient to prevent the bandage 100 from sticking to extraneous surfaces, at least one removable adhesive covering member is disposed on the inner surface 112 of the base substrate 101, such as a first adhesive covering liner 510 and a second adhesive covering liner 520. In at least one configuration, before being removed from the base substrate 101 the first adhesive covering liner 510 is disposed proximate to the first side edge 106 and the second adhesive covering liner 520 is disposed proximate to the second side edge 108. The first adhesive covering liner 510 and the second adhesive covering liner 520 are sized to at least completely cover the inner surface 112 of the base substrate 101, but can be sized larger to assist with removal of the first and second adhesive covering liners 510/520. In other configurations, the bandage 100 can include a single adhesive covering member or more adhesive covering members than those shown. In at least one configuration, the first adhesive covering liner 510 and the second adhesive covering liner 520 are sized such that they overlap each other proximate to the positions of the openings 132/134, as shown. In at least one configuration, the first and second adhesive covering liners 510/520 can include openings 532/534 therethrough that correspond to a position and size of the openings 132/134, respectively.

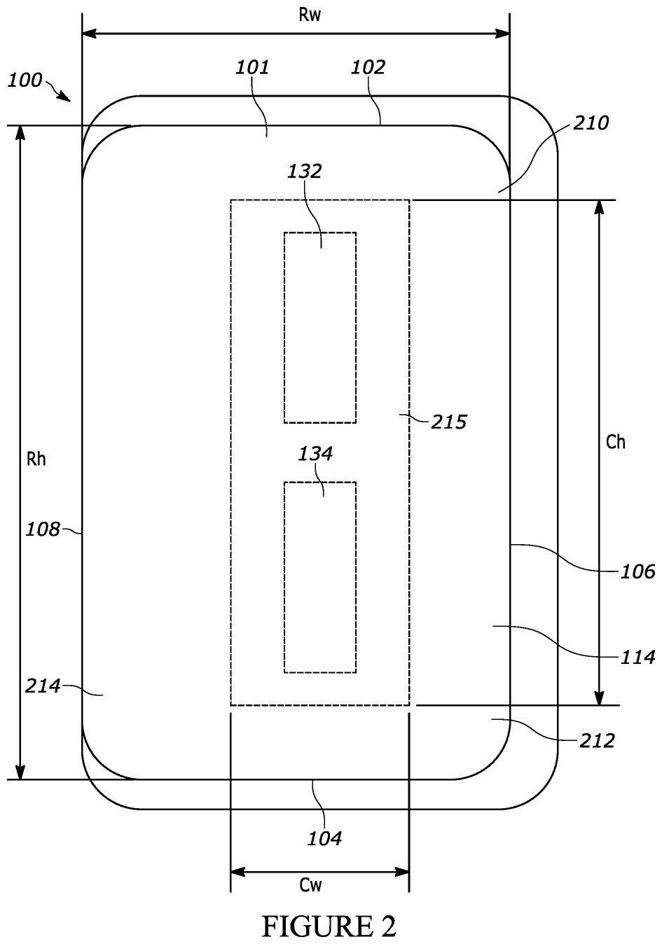

The bandage 100 further includes a replaceable treatment member 210 having a covering substrate, such as covering substrate 212, a wound covering 215 that is fixed (e.g., glued) to the covering substrate 212 and is positionable over the openings 132/134, as shown in FIG. 2, so that the wound covering 215 can come into contact with the wound of the patient during use of the bandage 100. The replaceable treatment member 210 further has a contact surface 633 (FIG. 6) that contacts the outer surface 114 of the base substrate 101 during use of the bandage 100, and a non-contact surface 214 that does not contact the bandage 100 during use of the bandage 100. In at least one configuration, the wound covering 215 is disposed offset on the replaceable treatment member substrate 212, as shown, although other locations are possible, and is approximately (+−5%) 2.75 inches in height Rh and approximately (+−5%) 1.8 inches in width Rw, although other shapes are possible such as circular, triangular, square, or any other shape. The covering substrate 212 is shown as being smaller in height and width than the base substrate 101, which allows for easy identification of edges of the covering substrate 212 during use, to assist with removal of the replaceable treatment member 210.

In at least one configuration, the wound covering 215 is a foam type of wound covering, although any type of wound covering is possible. In at least one configuration, the wound covering 215 is rectangular in shape, approximately (+−5%) 2.13 inches in height Ch and approximately (+−5%) 0.75 inches in width Cw, as shown, although other shapes are possible such as circular, triangular, square, or any other shape. In at least one configuration, another adhesive, a treatment member skin adhesive 634 (FIG. 6) is disposed on the contact surface 612 of the treatment member 210. The treatment member skin adhesive 634 is the type of adhesive that allows the covering substrate 212 to be removed from the base substrate 101 without removing the base substrate 101 from the skin of the patient, such as a silicone adhesive. Thus, the treatment member skin adhesive 634 is a weaker type adhesive than the skin adhesive 133. As patients routinely have bandages replaced during treatment/healing of a wound, the treatment member skin adhesive 634 can be removed from the base substrate 101 and replaced with a same or similar replaceable treatment member 210, all the while without disturbing the position of the base substrate 101 against the skin of the patient.

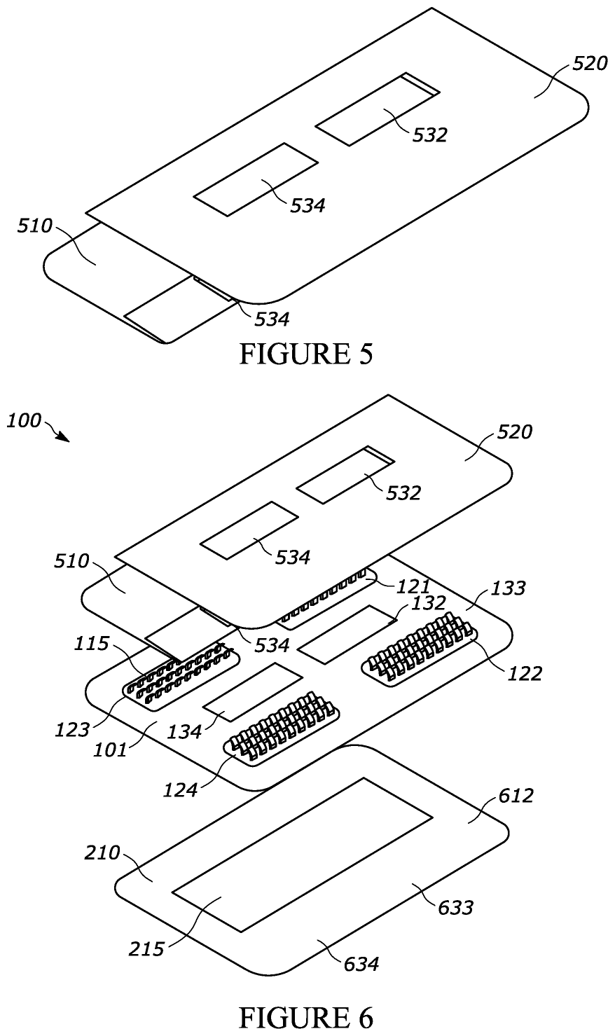

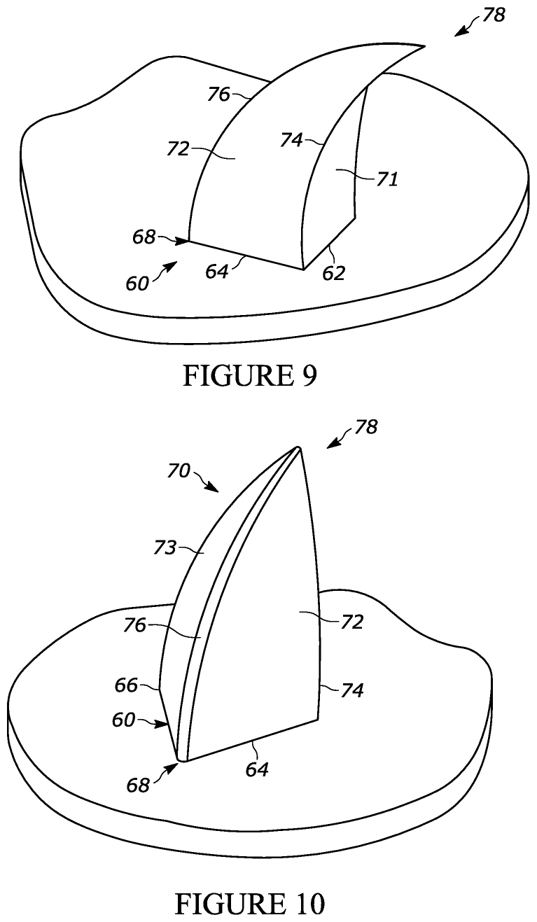

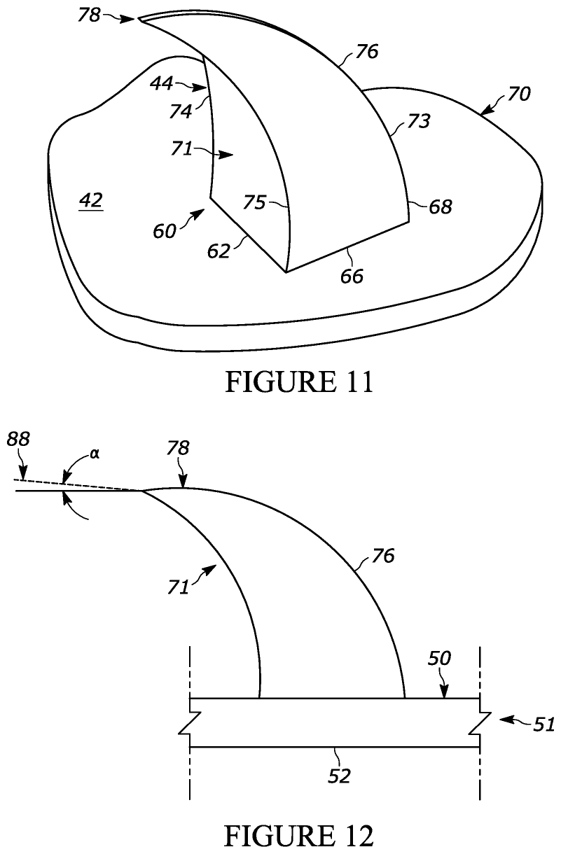

The plurality of gripping structures 115 can include the configuration shown in FIGS. 5-12. In at least one configuration, the mechanical coupling system 116 comprises a clear polymer, however, it is contemplated that translucent or opaque polymer constructions are likewise contemplated. The base member 110 comprises a lower surface 50, an upper surface 52, and an outer perimeter 54. The outer perimeter 54 is defined by leading edge 55, trailing edge 56, first side edge 57, and second side edge 58. The lower surface 50 and upper surface 52 cooperatively define the thickness 51 of the base member 110.

The shape of the bandage 100 is useful for holding a wound together, such as on a neck of a patient. However, such is an example, with the bandage 100 being useful for holding a wound together on other parts of the body, the bandage 100 being shaped to conform to different parts of the body. In at least one other configuration, the bandage 100 is rectangular, circular, triangular, or any other shape that conforms to a particular part of the body being treated.

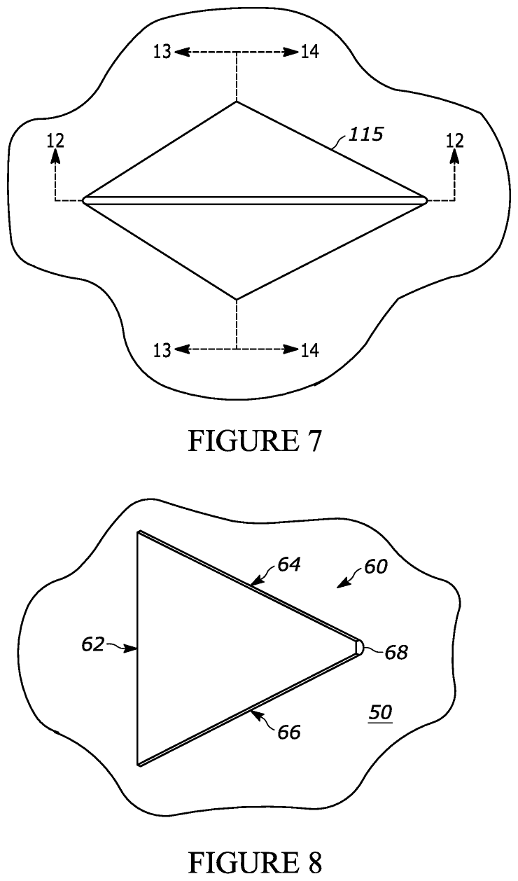

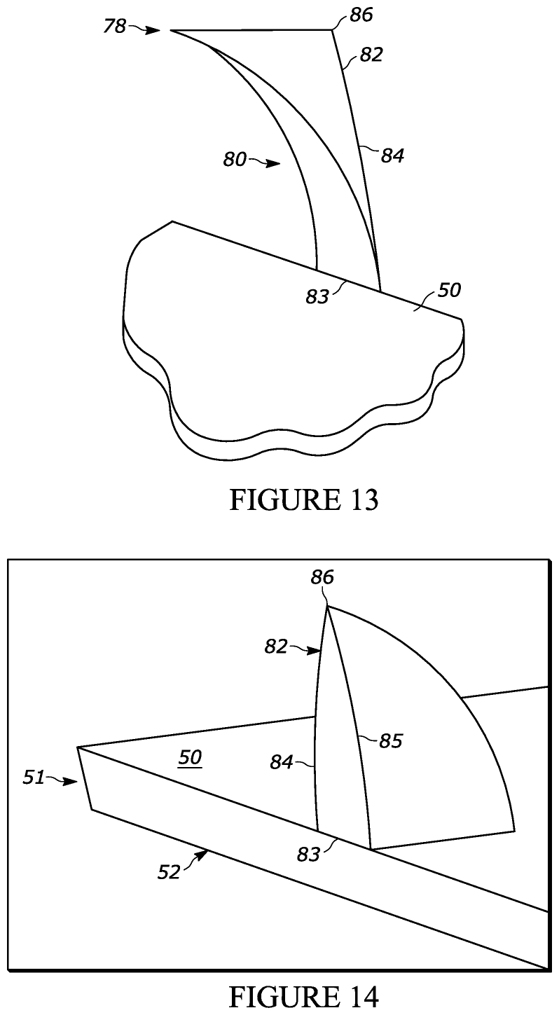

In at least one configuration, as shown in FIGS. 7-14, the gripping structures 115 comprise a base configuration 60 and upstanding structure 70. The base configuration 60 further comprises a leading edge 62, first rearward trailing edge 64, second rearward trailing edge 66, and rear intersection region 68. In the configuration shown, the base configuration 60 substantially defines a triangle, with the leading edge 62 being substantially parallel to the leading edge 55 of the base member 110, and generally perpendicular to the tip axis 188. The first and second rearward trailing edges 66, 68 together with the leading edge 55 define an isosceles triangle with the rear intersection region 68 defining the third point of the triangle. It has been found that a triangular cross-sectional configuration with the leading edge 55 being perpendicular to the direction of force (i.e., the biasing force of the bandage or other structure to direct the wound to closure) and inboard of the rear intersection region exhibits improved strength characteristics and retention characteristics that a configuration that wherein the base configuration 60 is flipped. In the configuration shown, the leading edge 62 is nominally 0.017 inches in width. The depth of the base structure, that is from the rear intersection region 68 to the leading edge 55 is nominally 0.0156 inches. As such, width is slightly greater than the depth, defining a generally isosceles triangle.

The upstanding structure 70 further comprises a front curved wall 71, first trailing wall 72, second railing wall 73, first upstanding edge 74, second upstanding edge 75, outer backbone 76, tip 78, and overhanging portion 80. The front curved wall 71 extends from the leading edge 62 of the base. Similarly, the first trailing wall 72 extends from the first rearward trailing edge 64 and the second trailing wall 73 extends from the second rearward trailing edge 66. From these walls, the edges are created. That is, the first upstanding edge 74 is defined by the intersection between the front curved wall 71 and the first trailing wall 72. The second upstanding edge 75 is defined by the intersection between the front curved wall 71 and the second trailing wall 73. The outer backbone 76 is defined as the intersection between the first trailing wall 72 and second trailing wall 73 and extends from the rear intersection region 68. The intersection of all the walls extending from the base configuration 60 defines the tip 78. The tip 78, due to the arcuate configuration of the outer back bone, and the first and second upstanding edges, defines a tip axis 88, which is generally controlled by the outer backbone the tip 78. In the configuration shown the tip axis is within a few degrees of being parallel to the lower surface 50 of the base member 110, and is preferably forms an angle a with a horizontal line intersecting at the tip of between −10° and 10°, and more preferably between −1° and 5°. It will be understood that in such a configuration, the axis is generally rather horizontal and facing in the direction of the force exerted by the inward biasing of the gripping structures.

In the configuration shown, the outer backbone is defined by multiple arcs, and in the configuration shown, three different arcuate configurations. The front curved wall is defined by multiple arcs, and in the configuration shown, two different arcuate configurations. The overhanging portion 80 begins noticeably following the interface plane 82, or the plane perpendicular to the base member 110 bisecting the upstanding structure 70 through the leading edge 62. Consequently, the base 83 of the interface plane 82 is colinear to the leading edge 62 of the base configuration 60. Following the interface plane 82 up the upstanding structure 70, the first edge 84 is the edge proximal to first side edge 24 and second edge 85 is the edge proximal to the second side edge 26. The intersection of these lines atop the upstanding structure 70 creates the top meeting region 86. In the configuration shown, the overall depth of the gripping structure, that is from the tip to the outer backbone at the base configuration 60 is nominally 0.029 inches. The height of the tip from the base is nominally 0.0230 inches. When compared to the thickness of the base, which is nominally 0.005 inches, the height is 4.6 times the thickness of the base. Whereas the base has a thickness which is similar to the thickness (or slightly thicker or thinner) than the epidermis, the tip is intended to extend beyond the epidermis and into the dermis of the patient or user. As such, it is desirable that the height is preferably 2 times the thickness of the base, and more preferably 4 or more times the thickness of the base. It will be understood that in other configurations, the upstanding structure 70 may be contained within the structure of the base configuration 60, and may not include an overhanging portion, while still including a tip as disclosed above that is directed toward the central region of the base substrate.

The overhang portions 80, that is the amount the tip is spaced apart from the leading edge is nominally 0.134 inches. Thus, the overhang portion extends beyond the footprint of the gripping structure (i.e., beyond the leading edge of the base configuration 60) by a distance that is approximately 85% of the depth of the gripping structure at the base configuration 60. Attention should be given to the overhand portions 80 of each of the upstanding structures 70, as its complexity begets a difficulty in classic molding mechanisms when molded with the base member 110. One skilled in the art may be familiar with the difficulty to mold materials with an overhand, or lip in the material, that can create a force to cement the molded object to the mold itself. The existence of these overhangs along the gripping structures, further added to the plurality of structures, creates a complex molding procedure. To mold the configuration shown, it is necessary to translate the mold in a direction that is perpendicular to the direction of removal of the member from the mold. That is, the molded member is first slid along the mold and then slid out of the mold. It may further be necessary to slightly rotate the molded member while being removed.

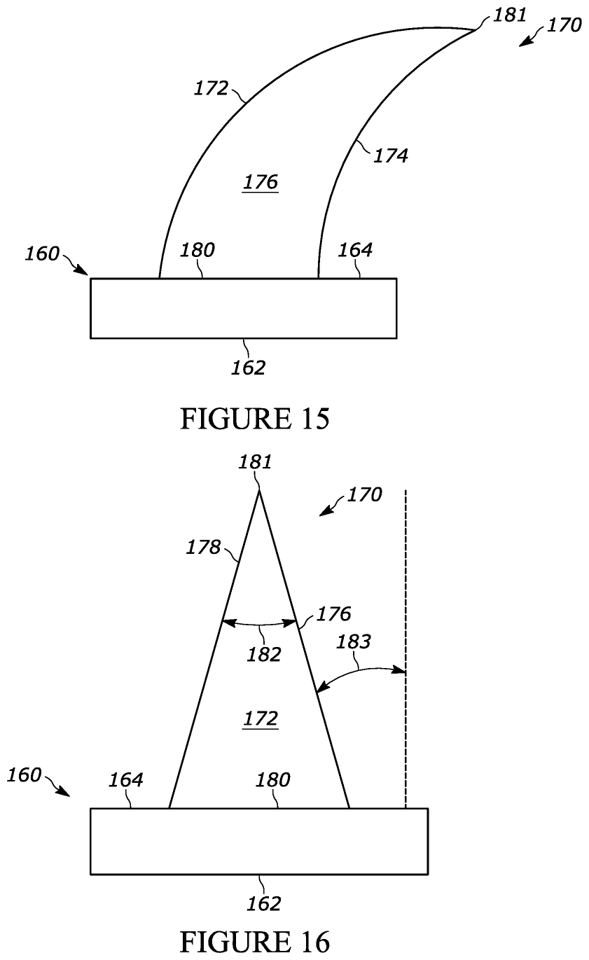

In an alternate configuration, the mechanical coupling members 121/122/123/124 can be mirror images of each other, and may comprise the alternate structure shown in FIGS. 15-17, collectively. In particular, the mechanical coupling members 121/122/123/124 can each comprise a base 160 and a spaced plurality of the gripping structure 170. The base 160 generally comprises a substantially planar member defining a lower surface 162, an upper surface 164 and outer perimeter 166. In the configuration shown, the base 160 has a thickness of 0.25 millimeters. When mounted in the operable configuration, the outer perimeter 166 defines a leading edge 167 and a trailing edge 168. In the configuration shown, the base 160 comprises a substantially uniform rectangular member with the longer sides thereof being the leading and trailing edges 167, 168, which are parallel to each other. Of course, other configurations are contemplated, including structures that have additional topographies, as discussed above for the structures shown in FIGS. 7-14.

The gripping structures 170 are spaced about the base 160 and extend from the upper surface 164 thereof. It will be understood that such structures are monolithic and conformed along with the base 160 to form a single substantially uniform configuration. The gripping structures 170 are generally (although not required to be) substantially identical in configuration. As such, a single gripping structure 170 is shown with the understanding that the remaining gripping structures are substantially identical.

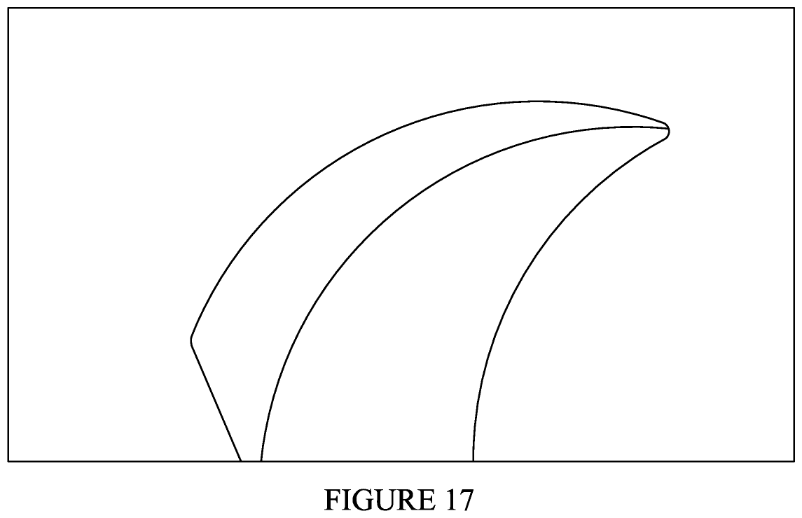

The gripping structure 170 includes leading surface 172, a trailing surface 174, first side surface 176 and second side surface 178. The gripping structure 170 at the base defines a base perimeter 180, which is substantially square. The four surfaces 172, 174, 176, and 178 cooperatively taper to an upper tip 181. The outer surfaces are angled inwardly at an angle 183 which, in the configuration shown is 12.02° with the included angel 182 of 24.04°. In the configuration shown, the height of the gripping structures is 0.700 mm. with the base 160 that is 0.426 mm square. The leading and trailing surfaces 172, 174 are each curved so that the upper tip 181 lies outside of the footprint defined by the base perimeter 180, with the first and second side surfaces 176, 178 being inclined at the angles identified above. Of course, variations are contemplated. It has been found that the curved surfaces of the leading and trailing surfaces 172, 174 greatly aids in the retention strength of the mechanical coupling members 121/122/123/124 as compared to straight or angled gripping structures. A magnification photograph of a gripping structure is shown in FIG. 17.

To manufacture the bandage 100 of the present configuration, it is contemplated that the base substrate 101, and the mechanical coupling system 116 can be provided on rolls with the components being joined together. Problematically, it may be difficult to provide a roll of the mechanical coupling system 116 without damaging the gripping structures 115. In such configurations, the mechanical coupling system 116 is provided on a roll encased, at least partially by a protective encasement. For example, a roll of what would become the mechanical coupling system 116 is provided, wherein one side forms the leading edge 55 of the mechanical coupling members 121/122/123/124, and the other side forms the trailing edge 56 of the mechanical coupling members 121/122/123/124.

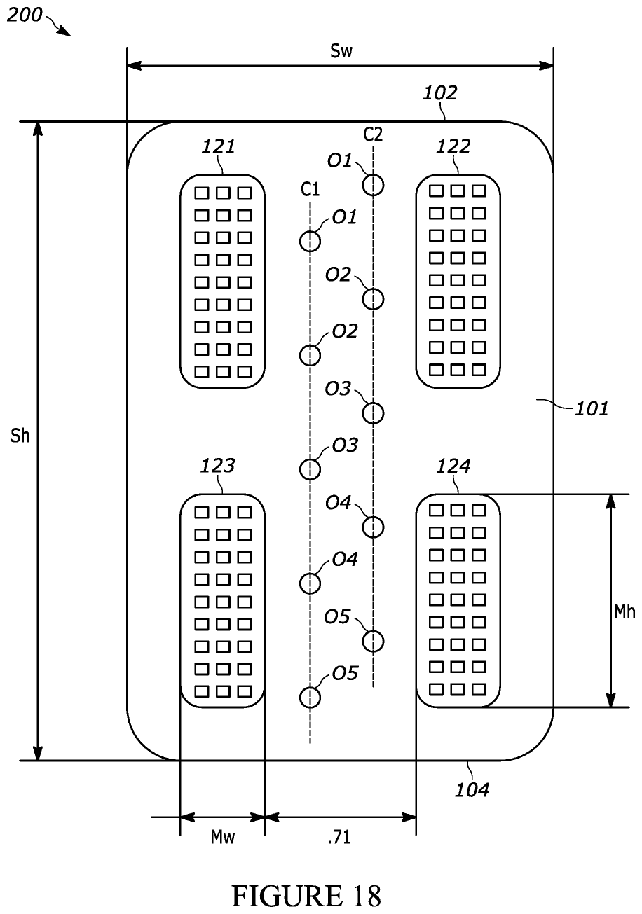

With reference to FIG. 18, another configuration for a bandage is disclosed, bandage 200. In this configuration, the bandage 200 is similar to bandage 100 and is similarly dimensioned. However, instead of including the openings 132 and 134 the bandage 200 includes a plurality of circular holes or circular openings disposed between the mechanical coupling members 121 and 122, and between mechanical coupling members 123 and 124. In this example, two columns of circular openings are disposed through the base substrate 101, a first column of openings C1 and a second column of openings C2. In at least one configuration, the first and second columns of openings C1, C2 each include five (5) circular openings O1, O2, O3, O4, O5 that are disposed in a straight line and are placed over a wound of a patient during use of the bandage 200, although more or less circular openings are possible.

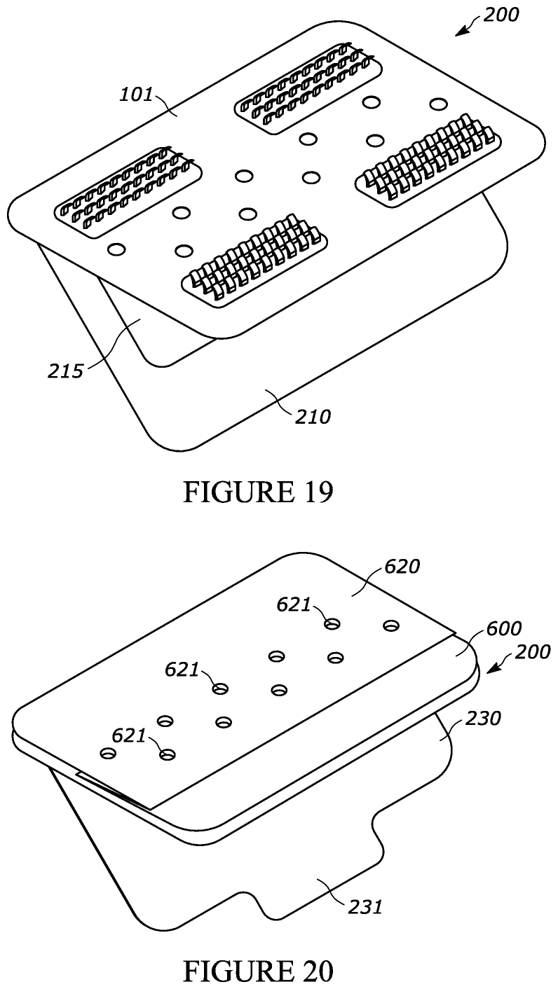

The first column of openings C1 is approximately a third of a distance between the mechanical coupling members 121 and 122, and between mechanical coupling members 123 and 124. The second column of openings C2 is approximately a two-thirds of a distance between the mechanical coupling members 121 and 122, and between mechanical coupling members 123 and 124. Although the first and second columns of openings C1, C2 can be otherwise disposed, in this example the first column of openings C1 is disposed closer to second end 104 of the base substrate 101 than the second column of openings C2, the first and second columns of openings C1, C2 thereby being staggered, although the first and second columns of openings C1, C2 can be non-staggered in at least one other configuration. In at least one configuration, the replaceable treatment member 210 shown in FIG. 2 can be coupled to the bandage 200 similarly to as being coupled the bandage 100 shown in FIG. 2, as shown in FIGS. 19 and 21.

In at least one configuration, instead of the replacement treatment member 210 being coupled to the bandage 200, a liner 230 can instead be coupled to the bandage 200, as shown in FIG. 20. The liner 230 can include a tab 231 to ease gripping of the liner 230. In at least one configuration, similar to the bandage 100 being used in conjunction with the first adhesive covering liner 510 and the second adhesive covering liner 520, the bandage 200 can be used with another first adhesive covering liner 610 and a second adhesive covering liner 620. As with the first and second adhesive covering liners 510, 520 including openings 532/534 therethrough that correspond to a position and size of the openings 132/134, respectively, the first and second adhesive covering liners 610, 620 include openings 621 that corresponding to a position and size of the circular openings O1, O2, O3, O4, O5 from the first and second columns of openings C1, C2.

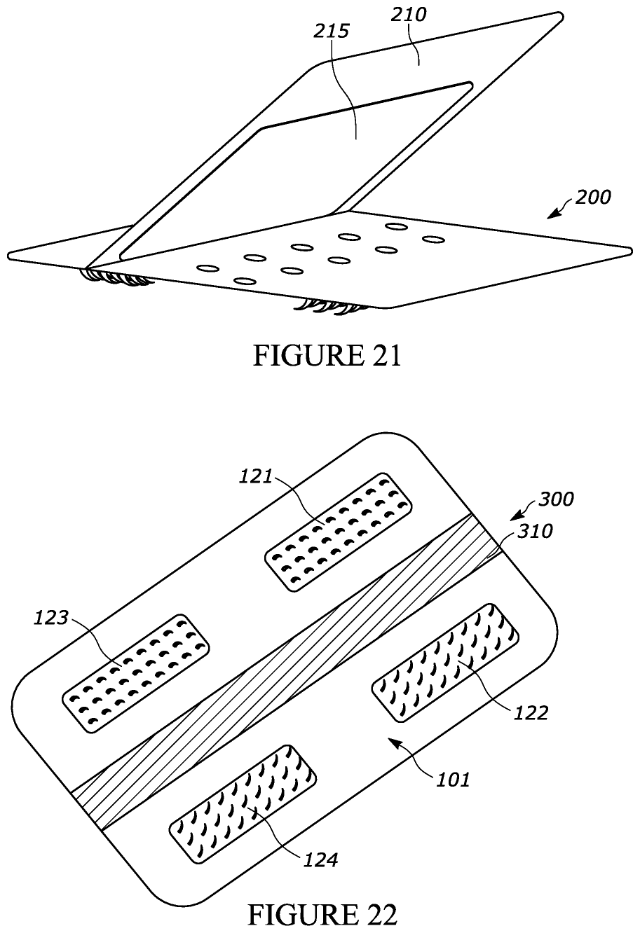

With reference to FIG. 22, yet another configuration of a bandage is disclosed, bandage 300. In this configuration, the bandage 300 is similar to bandage 100 and is similarly dimensioned. However, instead of including the openings 132 and 134 the bandage 300 includes another type of opening that is comprised of a mesh strip 310 through the base substrate 101 and centrally disposed between the mechanical coupling members 121 and 122, and between mechanical coupling members 123 and 124, the mesh strip 310 to be placed over a wound of a patient during use of the bandage 300. In at least one configuration, the mesh strip 310 can be approximately half as wide as the distance between the mechanical coupling members 121 and 122, and the distance between the mechanical coupling members 123 and 124. In this example, the mesh strip 310 extends from the first end 102 of the base substrate 110 to the second end 104 of the base substrate 101, although in at least one other configuration the mesh strip 310 can be shorter than that shown and not extend from the first end 102 of the base substrate 110 to the second end 104 of the base substrate 101.

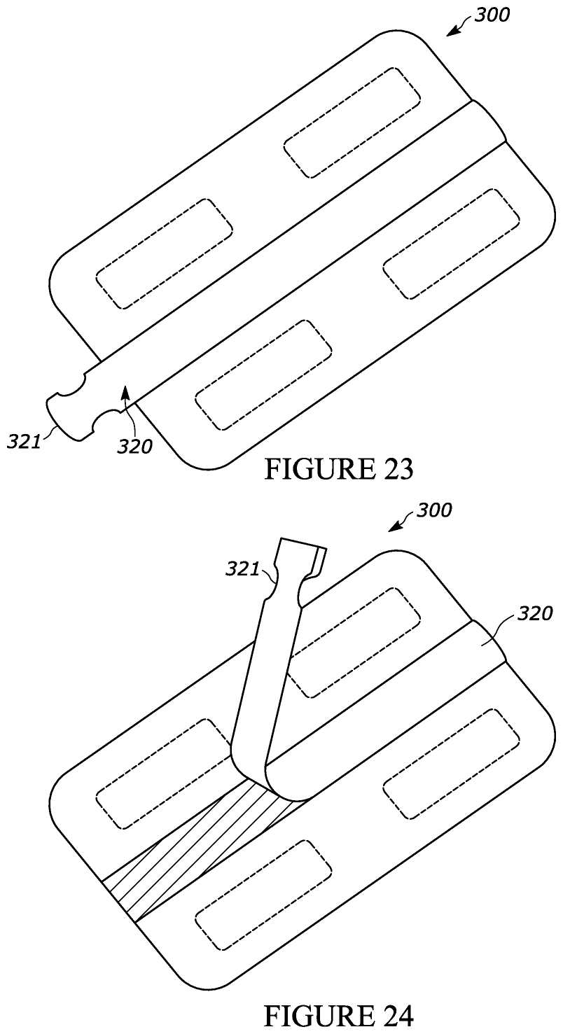

The mesh strip 310 provides fluid passage between the inner surface 112 and the outer surface 114 of the base substrate 101, similar to an opening providing such fluid passage, but at a slower rate. In at least one configuration, this mesh strip 310 is pre-impregnated at a time of manufacture of a bandage 700 (FIG. 28) with a cyanoacrylates skin adhesive 322 that is activated with air or moisture, although in at least one other configuration the cyanoacrylates skin adhesive 322 can be applied to the mesh strip 310 after the bandage 310 is applied to a wound. In at least one configuration, a back side liner 320 can be disposed (e.g., coupled) onto the mesh strip 310 to seal the mesh strip 310 and prevent the cyanoacrylates skin adhesive 322 from being activated to harden, the back side liner 320 being disposed on the outer surface 114 of the base substrate 101, as shown in FIG. 23. This back side liner 320 can be pulled back via a back side liner tab 321 to expose the mesh strip 310 and activate the cyanoacrylates skin adhesive 322 to harden.

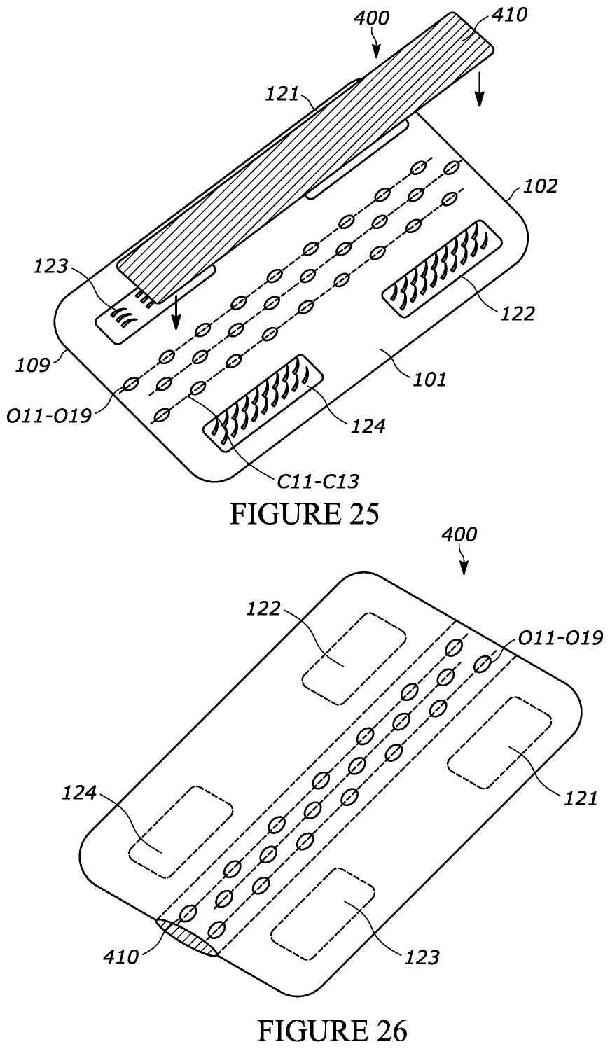

With reference to FIG. 25, even yet another configuration of a bandage is disclosed, bandage 400. The bandage 400 includes a similar configuration as bandage 200 in that a plurality of circular openings are disposed between the mechanical coupling members 121 and 122, and between mechanical coupling members 123 and 124. In this example, three columns of circular openings are disposed through the base substrate 101, a first column of openings C11, a second column of openings C12, and a third column of openings C13. In at least one configuration, the first, second, and third columns of openings C11, C12, C13 each include nine (9) circular openings O11, O12, O13, O14, O15, O16, O17, O19, O19 that are disposed in a straight line and extend from a first end 102 of the base substrate 101 to the second end 104 of the base substrate 101 that are placed over a wound of a patient during use of the bandage 400. In at least one other configuration, the bandage 400 can use more or less circular openings which do not extends to the first and second ends 102, 104 of the base substrate 101, and more or less columns of circular openings.

The bandage 400 further includes a mesh strip 410 that is disposed, e.g., fixed with an adhesive, over and onto the first, second, and third column of openings C11, C12, C13 within the substrate 101, the mesh strip 410 extending from the first end 102 to the second end 104 of the substrate 101. The mesh strip 410 is disposed onto a same side of the substrate 101 as the plurality of mechanical coupling members 121/122/123/124, that is the inner surface 114 of the substrate. FIG. 26 illustrates the bandage 400 from a perspective of the outer surface 114. Cyanoacrylates skin adhesive can be applied to the outer surface 114 over the openings C11, C12, C13 such that the cyanoacrylates skin adhesive flows through the first, second, and third column of openings C11, C12, C13 and into the mesh strip 410.

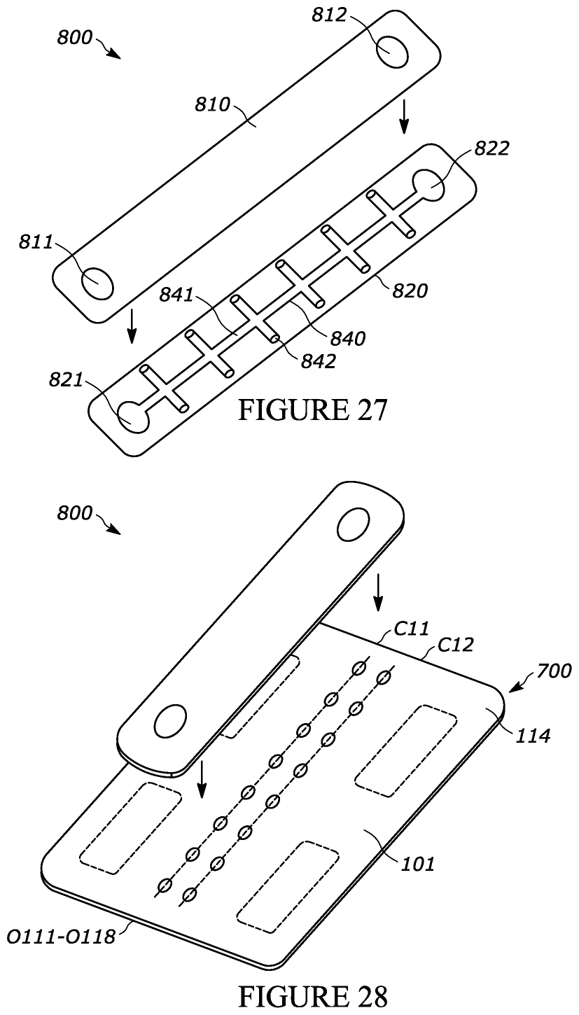

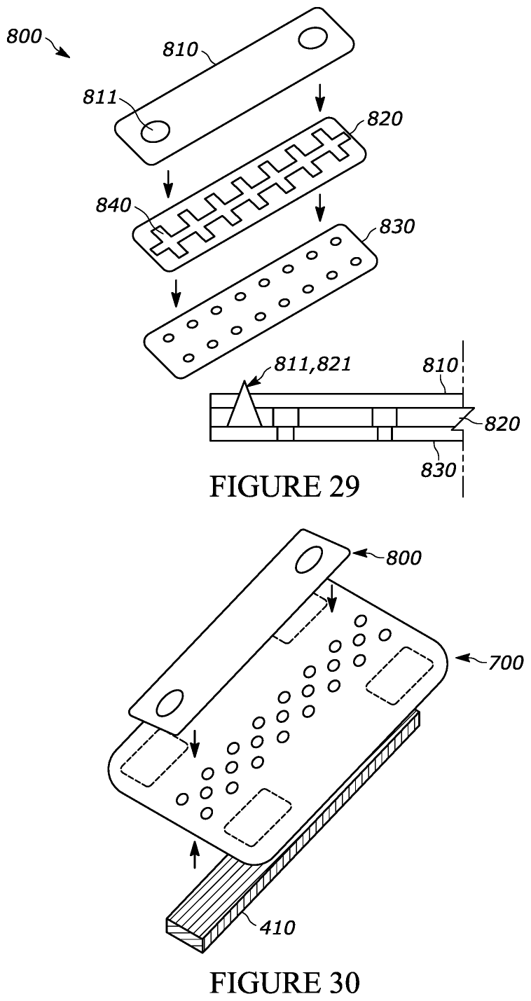

With reference to FIG. 27, an adhesive delivery system, such as adhesive delivery system 800 that can be coupled (e.g., via adhesive) to the outer surface 114 of the base substrate 101 of a bandage 800 (FIG. 28), the bandage 700 thereby including the adhesive delivery system 800. The adhesive delivery system 800 delivers a skin adhesive to openings within the bandage 700. The adhesive delivery system 800 includes a cover film 810 disposed onto (e.g. such as via adhesive) an intermediate film 820, the intermediate film 820 being disposed onto a base film 830 (FIG. 29) together forming a laminate, and a skin adhesive 830 therebetween, such as a cyanoacrylates skin adhesive. The cover film 810 is rectangular in shape, approximately sized and shaped to correspond to one more bandage configuration, bandage 700.

The cover film 810 includes blister indicators 811, 812 disposed proximate to distal ends of the cover film 810. The intermediate film 820 includes blisters 821, 822 disposed to correspond at locations of blister indicators 811, 812. The blisters 821, 822 are reservoirs to store an adhesive, such as cyanoacrylates skin adhesive. The blisters 821, 822 are each coupled to a vein network 840 that includes tubes or veins 842 to distribute the skin adhesive 830 to the bandage 700 at locations that correspond to openings within the bandage 700, as discussed below. The blisters 821, 822 are squeezed by a user to push the skin adhesive 830 out of the blisters 821, 822 and into the veins 842, at which point the skin adhesive 830 exits the adhesive delivery system 800 at openings 842 through the base film 830, the openings 842 at least approximately corresponding to locations of the openings through the bandage 700.

Instead of including the openings 132 and 134, the bandage 700 includes a plurality of circular holes or circular openings disposed between the mechanical coupling members 121 and 122, and between mechanical coupling members 123 and 124. In this example, two columns of circular openings are disposed through the base substrate 101, a first column of openings C111 and a second column of openings C112. In at least one configuration, the first and second columns of openings C1, C2 each include eight (8) circular openings O111, O112, O113, O114, O115, O116, O117, O118 that are disposed in a straight line and are placed over a wound of a patient during use of the bandage 700. In at least one configuration of the adhesive delivery system 600 and bandage 700 combination can be used with the mesh strip 410 that is disposed (e.g., via adhesive) onto the inner surface 112 of the base substrate 101, as shown in FIG. 30.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A bandage, comprising:
a base substrate including an inner surface, an outer surface, a first side edge, and a second side edge;
a mechanical coupling system including a first mechanical coupling member disposed proximate to the first side edge of the base substrate and a second mechanical coupling member disposed proximate to the second side edge of the base substrate the first and second mechanical coupling members each includes a plurality of gripping structures to hold a wound together; and
an opening disposed through the base substrate and between the first and second mechanical coupling members.

2. The bandage according to claim 1, further comprising a third mechanical coupling member disposed proximate to the first side edge of the base substrate and a fourth mechanical coupling member disposed proximate to the second side edge of the base substrate.

3. The bandage according to claim 1, wherein the opening through the base substrate is comprised of a mesh strip.

4. The bandage according to claim 3, wherein the mesh strip is pre-impregnated with a skin adhesive, the bandage further comprising a back side liner disposed on the mesh strip to prevent the pre-impregnated skin adhesive from being activated.

5. The bandage according to claim 1, wherein the opening through the base substrate includes a first column of a plurality of openings and a second column of the plurality of openings.

6. The bandage according to claim 5, wherein the first column of the plurality of openings is staggered with respect to the second column of the plurality of openings.

7. The bandage according to claim 5, wherein the opening through the base substrate includes a third column of the plurality of openings.

8. The bandage according to claim 1, wherein the opening is rectangular in shape.

9. The bandage according to claim 1, wherein the opening is circular in shape.

10. The bandage according to claim 1, further comprising a replaceable treatment member coupled to the base substrate, the replaceable treatment member including a wound covering that corresponds to the opening, the wound covering to cover the wound.

11. The bandage according to claim 1, further comprising a skin adhesive disposed on an outer surface of the base substrate.

12. The bandage according to claim 11, further comprising an adhesive covering liner disposed on the base substrate.

13. The bandage according to claim 1, wherein the first and second mechanical coupling members each includes rows of the plurality of gripping structures.

14. The bandage according to claim 1, wherein the base substrate is approximately 2 inches in width and 3 inches in height.

15. The bandage according to claim 1, wherein the opening includes a plurality of columns of openings, the bandage further comprising a mesh strip disposed on the plurality of openings and on a same side of the base substrate as the mechanical coupling system.

16. The bandage according to claim 1, further comprising an adhesive delivery system to store a skin adhesive and deliver the skin adhesive to the openings through the base substrate.

17. A method of coupling a bandage to a skin of a patient, the method comprising:
placing an opening of a bandage over a wound, the bandage comprising:
a base substrate including an inner surface, an outer surface, a first side edge, and a second side edge;
a mechanical coupling system including a first mechanical coupling member disposed proximate to the first side edge of the base substrate and a second mechanical coupling member disposed proximate to the second side edge of the base substrate, the first and second mechanical coupling members each includes a plurality of gripping structures to hold a wound together; and
the opening being through the base substrate and between the first and second mechanical coupling members.

18. The method according to claim 17, wherein the bandage further comprises a third mechanical coupling member disposed proximate to the first side edge of the base substrate and a fourth mechanical coupling member disposed proximate to the second side edge of the base substrate.

19. The method according to claim 17, wherein the opening through the base substrate is comprised of a mesh strip.

20. The method according to claim 17, wherein the mesh strip is pre-impregnated with a skin adhesive, the bandage further comprising a back side liner disposed on the mesh strip to prevent the pre-impregnated skin adhesive from being activated, the method further comprising pulling back the back side liner to activate the pre-impregnated skin adhesive.
